(19) United States
(12) Patent Application Publication
(10) Pub. No.: US 2025/0169732 A1
(43) Pub. Date: May 29, 2025

(54) PSYCHOLOGICAL STATE ESTIMATION DEVICE AND PSYCHOLOGICAL STATE ESTIMATION METHOD

(71) Applicant: OMRON Corporation, Kyoto-shi, Kyoto (JP)

(72) Inventors: Ayana TSUJI, Kyoto-shi, KYOTO (JP); Masahiko OGAWA, Kyoto-shi, KYOTO (JP); Takenori HATSUDA, Kyoto-shi, KYOTO (JP); Shoya MURAKAMI, Kyoto-shi, KYOTO (JP)

(21) Appl. No.: 18/841,871

(22) PCT Filed: Jan. 24, 2023
(86) PCT No.: PCT/JP2023/002079

§ 371 Date: Aug. 27, 2024

(30) Foreign Application Priority Data
Mar. 9, 2022 (JP) 2022-036588

(51) Int. Cl. A61B 5/16; A61B 5/00

(52) U.S. Cl. CPC A61B 5/165; A61B 5/0077; A61B 5/742

(57) ABSTRACT

A psychological state estimation device includes a display section configured to display a predetermined image, an imaging section configured to image a face of an observer viewing the predetermined image displayed by the display section, a facial expression estimation section configured to estimate a facial expression of the observer from an image of the face imaged by the imaging section, and a state estimation section configured to estimate a psychological state of the observer based on a change in the facial expression of the observer viewing the predetermined image, the facial expression being estimated by the facial expression estimation section.

### TECHNICAL FIELD

The present invention relates to an estimation technology of a psychological state.

### BACKGROUND ART

In modern society in which overwork death and accidents due to fatigue of workers, mental health problems, and the like are social problems, it is important to visualize and manage psychological states such as fatigue and stress. In addition, there are some working methods such as remote work in which it is more difficult to grasp the psychological state of the worker than in related art, and a technology capable of easily estimating the psychological state in various environments is required.

As the technology for estimating the psychological state, for example, there is a method of using biological information such as a heartbeat and brain waves. However, in the method of using the biological information, since a dedicated measuring instrument may be used, it is difficult to easily estimate the psychological state at home or the like.

As another technology for estimating the psychological state, there is a method of using a face image of a user. For example, in the technology disclosed in Patent Document 1, a degree of psychogenic plague is determined from the face image of the user by using a diagnostic matrix obtained by quantifying knowledge of experts. In the technology disclosed in Patent Document 2, a feature related to a relative position of a part of the face or the like is calculated from the face image of the user, and an emotion is estimated.

### CITATION LIST

### Patent Literature

Patent Document 1: Japanese Unexamined Patent Publication No. 2006-305260
Patent Document 2: Japanese Unexamined Patent Publication No. 2021-024378

### SUMMARY OF THE INVENTION

### Problems to be Solved by the Invention

However, in the technology disclosed in Patent Document 1, since individual differences in which the psychological state appears in a facial expression are not considered, the estimation accuracy of the psychogenic plague may be lowered. In the technology disclosed in Patent Document 2, since whether or not the estimated emotion coincides with the emotion felt by the user himself or herself is not considered, the estimation accuracy of the emotion may be lowered.

The present invention has been made in view of the above problems, and an object thereof is to easily and accurately estimate a psychological state of a user.

### Means for Solving the Problem

A first aspect of the present invention provides a display section configured to display a predetermined image, an imaging section configured to image a face of an observer viewing the predetermined image displayed by the display section, a facial expression estimation section configured to estimate a facial expression of the observer from an image of the face imaged by the imaging section, and a state estimation section configured to estimate a psychological state of the observer based on a change in the facial expression of the observer viewing the predetermined image, the facial expression being estimated by the facial expression estimation section.

The predetermined image may be a still image or a moving image. In addition, the predetermined image may be, for example, an image that induces facial expression imitation, such as an image in which a person shows a certain emotion. The face image may show the face of the observer (user), and may include a head, a neck, an upper body, and the like. In the estimation of the facial expression, a plurality of facial expressions (for example, neutral, joy, surprise, sadness, anger, and the like) may be estimated, or one facial expression (for example, how many percent a degree of joy is) may be estimated. Similarly, in the estimation of the psychological state, a plurality of psychological states may be estimated, or one psychological state may be estimated. Since not a dedicated measuring instrument or the like for estimating the psychological state but the face image of the observer viewing the predetermined image is used, the psychological state estimation device can easily estimate the psychological state. In addition, since the appearance of the emotion unconsciously, which is a change in the facial expression when the observer looks at the predetermined image, is grasped for estimation, the psychological state estimation device can accurately estimate the psychological state. Accordingly, according to this configuration, the psychological state of the user can be easily and accurately estimated.

The state estimation section may be configured to analyze a correlation between the change in the facial expression and the psychological state, and estimate the psychological state of the observer. The change in the facial expression may be a change in all the periods in which the face image is acquired, or may be a specific period among the periods in which the face image is acquired. According to this configuration, the psychological state can be estimated from the change in the facial expression of the observer viewing the predetermined image.

The state estimation section may be configured to analyze the correlation for each observer. There are individual differences in a degree of appearance of a facial expression in an emotion. Accordingly, according to this configuration, the psychological state can be accurately estimated in consideration of the individual difference for each observer.

The facial expression estimation section may be configured to calculate a facial expression score obtained by quantifying the facial expression. According to this configuration, the psychological state can be estimated by using the change in the calculated facial expression score.

The state estimation section may be configured to estimate the psychological state based on a temporal change of the facial expression score in a predetermined period. The predetermined period may be, for example, all the periods in which the face image is acquired, or may be specific periods such as periods before and after the predetermined image is displayed. For example, the temporal change may be calculated from the feature of the waveform indicated by the time-series data of the facial expression score. According to this configuration, it is possible to determine whether or not the facial expression change is poor as compared with normal times, for example, from the temporal change of the facial expression score.

The state estimation section may be configured to estimate the psychological state based on an average value of the facial expression scores in periods corresponding to display periods in which the predetermined image is displayed. The periods corresponding to the display periods are, for example, periods in which the face image of the observer at the time of facial expression imitation is regarded as being acquired. The average value of the facial expression scores may be an average value in periods corresponding to a plurality of display periods, or may be an average value in a period corresponding to one display period. According to this configuration, from a change in the average value of the facial expression scores at the time of facial expression imitation, for example, it is possible to determine whether or not the facial expression change is poor as compared with normal times.

The imaging section may be configured to image the face in periods corresponding to display periods in which the predetermined image is displayed and the face in periods corresponding to non-display periods in which the predetermined image is not displayed, and the state estimation section is configured to estimate the psychological state based on a change between facial expression scores in the periods corresponding to the display periods and facial expression scores in the periods corresponding to the non-display periods. The change between the facial expression scores in the periods corresponding to the display period and the facial expression scores in the periods corresponding to the non-display periods may be calculated by using, for example, an average value or a variance. According to this configuration, it is possible to determine whether or not the facial expression change is poor as compared with normal times, for example, from the change in the facial expression scores in the periods corresponding to the display periods and the facial expression scores in the periods corresponding to the non-display periods.

The display section may be configured to display different images at predetermined times. For example, in a case where the predetermined image is displayed for 10 seconds, the psychological state estimation device may display a different image every 2 seconds. In addition, in a case where the predetermined image is displayed after a lapse of a predetermined time (for example, after a lapse of two hours), the psychological state estimation device may display an image different from the image displayed in the previous display period, for example. According to this configuration, it is possible to avoid a decrease in the estimation accuracy of the psychological state such as a change in a degree of facial expression imitation due to the observer being familiar with the displayed image.

The predetermined image may include a positive image for inducing a positive emotion to the observer and a negative image for inducing a negative emotion to the observer. The positive image and the negative image may be the same image regardless of the observer, or may be different images in accordance with the preference of the observer. According to this configuration, it is possible to determine whether or not the degree of facial expression imitation for a specific facial expression has changed.

The psychological state estimation device may further include an output section configured to output information indicating one or more emotions based on the psychological state estimated by the state estimation section. The information indicating one or more emotions may be output in such a manner that the psychological state of the observer can be grasped. The output destination may be a device used by the observer or a device different from the device used by the observer. According to this configuration, the observer, the supervisor of the observer, or the like can know the estimation result of the psychological state of the observer.

A second aspect of the present invention provides a psychological state estimation method including a display step of displaying a predetermined image, an imaging step of imaging a face of an observer viewing the predetermined image displayed in the display step, a facial expression estimation step of estimating a facial expression of the observer from an image of the face imaged in the imaging step, and a state estimation step of estimating a psychological state of the observer based on a change in the facial expression estimated in the facial expression estimation step.

A third aspect of the present invention provides a program for causing a computer to execute steps of the psychological state estimation method.

### Effect of the Invention

According to the present invention, it is possible to easily and accurately estimate the psychological state of the user.

### BRIEF DESCRIPTION OF THE DRAWINGS

FIG. 1 is a diagram illustrating a usage example of a state estimation device according to an embodiment of the present invention.

FIG. 2 is a diagram illustrating details of a configuration of the state estimation device.

FIG. 3 is a table representing an example of a facial expression estimation result.

FIG. 4 is a flowchart illustrating estimation processing of a psychological state.

FIG. 5 is a diagram illustrating an example of a change in a facial expression score.

### MODE FOR CARRYING OUT THE INVENTION

### Application Example

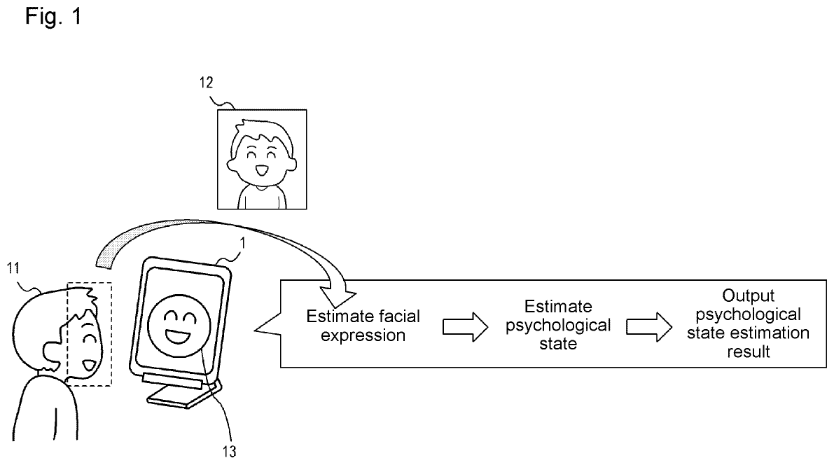

First, an example of a scene to which the present invention is applied will be described. FIG. 1 is a diagram illustrating a usage example of a state estimation device according to an embodiment of the present invention.

A state estimation device 1 is an electronic device (psychological state estimation device) that estimates a psychological state of a user (observer) 11. In FIG. 1, a user 11 views a facial expression image 13 (predetermined image), which is an image inducing facial expression imitation, displayed on a display (display device) of the state estimation device 1. Note that the facial expression imitation is a phenomenon in which the user unconsciously and reflectively performs a facial expression similar to a facial expression of the other person by looking at the facial expression of the other person. The facial expression imitation is used, and thus, it is possible to estimate the psychological state without giving the user 11 stress such as making a facial expression for estimating the psychological state, for example. The facial expression image 13 includes, for example, an image for inducing a positive emotion to the user and an image for inducing a negative emotion. The state estimation device 1 estimates a change in facial expression from a face image 12 obtained by imaging the face of the user 11 viewing the facial expression image 13, and estimates the psychological state.

A function and specification of a client program for estimating the psychological state are arbitrary, but in the present application example, a program (hereinafter referred to as “state estimation software”) for outputting an estimation result of the psychological state of the user is exemplified. First, the user 11 activates the state estimation software of the state estimation device 1. Then, the state estimation device 1 (specifically, a CPU operating according to the state estimation software) displays the facial expression image 13 on the display at a predetermined time.

The state estimation device 1 estimates a facial expression of the user 11 from the face image 12 when the facial expression image 13 is presented to the user 11. The state estimation device 1 analyzes a correlation between a change in facial expression and a psychological state for each individual to estimate the psychological state. The state estimation device 1 can estimate the psychological state with higher accuracy in consideration of an individual difference in which the psychological state appears in the facial expression by analyzing the correlation between the change in the facial expression and the psychological state for each individual to estimate the psychological state.

The state estimation device 1 outputs the estimation result of the psychological state. For example, the state estimation device 1 may output a degree of one emotion such as a “degree of vivacious of 90%”, or may output degrees of a plurality of emotions such as “degree of vivacious of 70%, degree of stress of 30%”. In addition, the state estimation device 1 may output two patterns such as “normal/high stress” and “positive/negative”. In addition, the estimation result may be output to a device different from a device used by the user 11, an external server, or the like. For example, the estimation result is also transmitted to a device of a supervisor, and thus, the supervisor can easily grasp whether or not a psychological state of a subordinate is good.

### (Configuration of State Estimation Device)

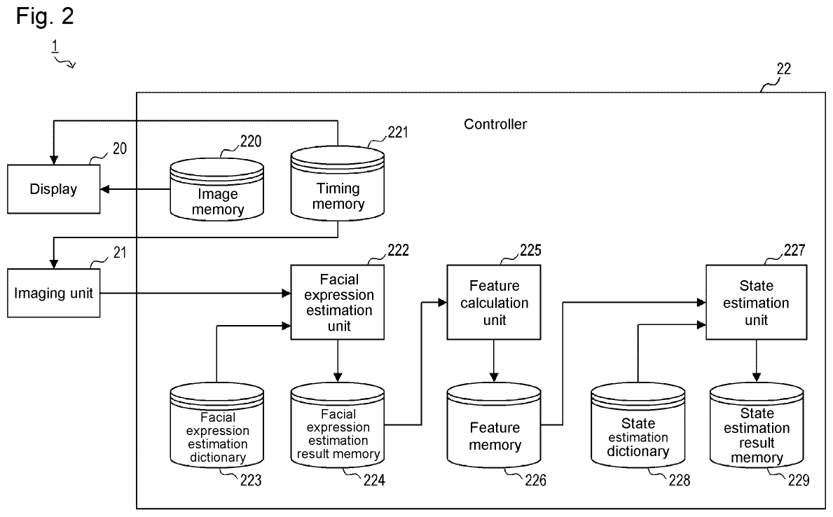

Next, a specific configuration example of the state estimation device 1 of the embodiment will be described with reference to FIG. 2.

The state estimation device 1 includes a display (display section) 20, an imaging unit (imaging section) 21, and a controller 22. The controller 22 includes an image memory 220, a timing memory 221, a facial expression estimation unit 222, a facial expression estimation dictionary 223, a facial expression estimation result memory 224, a feature calculation unit 225, a feature memory 226, a state estimation unit 227, a state estimation dictionary 228, and a state estimation result memory 229.

The display 20 displays a predetermined image (a facial expression image that is an image inducing facial expression imitation) and the like stored in the image memory 220 at a timing stored in the timing memory 221. For example, a liquid crystal display, an organic EL display, or the like can be used as the display 20.

The imaging unit 21 generates and outputs image data by photoelectric conversion. For example, the imaging unit 21 includes an imaging element such as a charge-coupled device (CCD) and a complementary metal oxide semiconductor (CMOS). The imaging unit 21 images the face image of the user at the timing stored in the timing memory 221, and outputs the imaged face image to the facial expression estimation unit 222. Note that the imaging unit 21 images the face image not only in a period corresponding to a display period in which the facial expression image is displayed (presented) but also in a period corresponding to a non-display period in which the facial expression image is not displayed.

The image memory 220 stores a predetermined image. The predetermined image stored in the image memory 220 may be an image acquired from an outside of the state estimation device 1 via an interface or the image acquired by the imaging unit 21.

The timing memory 221 stores a display timing at which a predetermined image is displayed on the display 20 and an imaging timing at which the imaging unit 21 images the face image of the user.

The facial expression estimation unit 222 estimates the facial expression of the user by using the face image acquired by the imaging unit 21 and the facial expression estimation dictionary 223. The facial expression estimation unit 222 estimates the facial expression by using an image feature that is a feature such as a brightness difference and a shape of a part constituting the face. The image feature is, for example, a Haar-like feature obtained from a local brightness difference or a Hog feature obtained from a distribution of a local luminance gradient direction, but is not limited thereto. The facial expression estimation unit 222 may estimate the facial expression by using a generally known technology for determining the facial expression. The facial expression estimation unit 222 outputs an estimation result of the facial expression to the facial expression estimation result memory 224.

The facial expression estimation dictionary 223 is a dictionary in which a correlation between the image feature and the facial expression is trained by using machine learning or the like. The machine learning is, for example, a cascade classifier or a convolutional neural network (CNN), but is not limited thereto.

In the embodiment, the facial expression estimation unit 222 calculates a facial expression score obtained by quantifying the facial expression as a measure expressing the facial expression. For example, the facial expression score is calculated from proportions of “neutral, joy, surprise, sadness, and anger” estimated by the facial expression estimation unit 222 from the acquired face image. Note that the facial expression estimation unit 222 may calculate a part of the facial expressions of “neutral, joy, surprise, sadness, and anger” or may calculate the facial expression including other facial expressions.

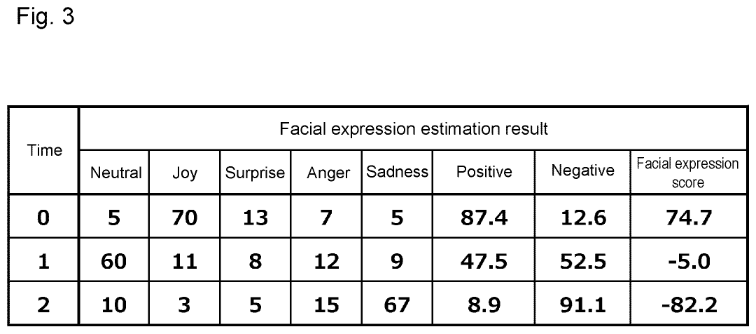

A specific description will be given with reference to FIG. 3. FIG. 3 is a table representing examples of the facial expression estimation result. The facial expression estimation unit 222 calculates scores of positive facial expressions (joy and surprise) and negative facial expressions (anger and sadness) excluding neutral among the facial expressions. The facial expression estimation unit 222 sets, as the facial expression score, a total value calculated with the positive facial expression as a positive number and the negative facial expression as a negative number. For example, a score Sp of the positive facial expression, a score Sn of the negative facial expression, and a facial expression score Se at time 0 are calculated by the following Expressions 1 to 3.

Sp=(70+13)/(70+13+7+5)×100=87.4 (Expression 1)

Sn=(7+5)/(70+13+7+5)×100=12.6 (Expression 2)

Se=Sp−Sn=87.4−12.6=74.7 (Expression 3)

The description returns to FIG. 2. The facial expression estimation result memory 224 stores the estimation result of the facial expression output by the facial expression estimation unit 222. The facial expression estimation result memory 224 stores information indicating when and what kind of facial expression the user has. Note that the facial expression estimation result memory 224 may store only the facial expression score or may store the proportion of each expression in addition to information regarding the time.

The feature calculation unit 225 calculates a score feature that is a feature related to a change in the facial expression of the user. For example, the feature calculation unit 225 calculates a score feature from the amount of change in the facial expression score, and outputs the calculated result to the feature memory 226. Note that details of the score feature used by the feature calculation unit 225 will be described later. The feature memory 226 stores the score feature output from the feature calculation unit 225.

The state estimation unit 227 analyzes the correlation between the change in the facial expression and the psychological state to estimate the psychological state of the user. For example, the state estimation unit 227 may estimate the psychological state by using a result of training in advance for each individual (user). For example, the state estimation unit 227 estimates the psychological state of the user by using the state estimation dictionary 228. The state estimation dictionary 228 is a dictionary in which the correlation between the psychological state and the score feature is trained for each individual. When the correlation between the psychological state and the score feature is trained in advance, the state estimation dictionary 228 may define a current psychological state of the user from, for example, an answer of the user to a questionnaire. The questionnaire may include a plurality of question groups or may accept an answer to one question. For example, a question group of “is stress high?” and “is it vivacious?” may be answered with “Yes/No”. In addition, in response to the question “What is your stress level today?”, the user may input a stress level as a numerical value.

In addition, for example, the state estimation unit 227 may estimate the psychological state without performing training in advance for each individual. For example, the state estimation unit 227 may estimate the psychological state by using a general-purpose dictionary (for example, a dictionary in which a correlation between a psychological state defined from answers of a large number of subjects to a questionnaire and a score feature is trained) created from experimental results by the large number of subjects. In addition, the state estimation unit 227 may estimate the psychological state by rule-based inference. In the rule-based inference, the state estimation unit 227 may estimate the psychological state based on a rule created from general knowledge that the stress level is high when the facial expression change is poor. As described above, the state estimation unit 227 may estimate the psychological state by using the result of training for each individual, or may estimate the psychological state without performing training for each individual.

The psychological state and the score feature are analyzed for each individual, and thus, the state estimation unit 227 can estimate the psychological state in consideration of an individual difference in which the psychological state appears in the facial expression. The state estimation unit 227 outputs the estimated psychological state to the state estimation result memory 229. The state estimation result memory 229 stores the estimation result of the psychological state output by the state estimation unit 227.

The state estimation device 1 includes, for example, a computer including hardware resources such as a central processing unit (CPU), a memory, a storage, and a display device. Blocks 20 to 22 and 220 to 229 illustrated in FIG. 2 are implemented by the CPU loading a program (operating system, state estimation software, and the like) stored in the storage in the memory and executing the program. However, the configuration of the state estimation device 1 is not limited thereto. For example, some or all of functions provided by the state estimation device 1 may be implemented by dedicated hardware such as ASIC or FPGA. In addition, a part of the functions of the state estimation device 1 may be executed by a cloud server.

### (Estimation Processing)

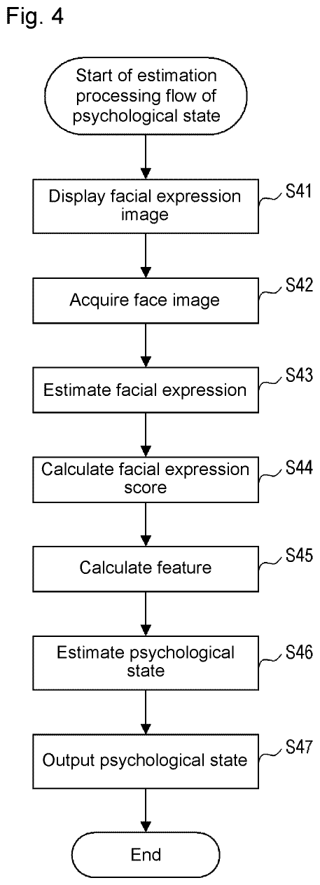

Next, an estimation processing flow of the psychological state will be described with reference to FIG. 4. FIG. 4 is a flowchart illustrating the estimation processing of the psychological state.

In step S41, the state estimation device 1 displays the facial expression image that is the image inducing the facial expression imitation on the display. The facial expression image includes a positive image (for example, a picture of a smiling face) that induces a positive emotion in the user and a negative image (for example, a picture of a crying face) that induces a negative emotion in the user. Note that an image to be displayed preferably has randomness. For example, when an image to be displayed as the positive image is the same image every time, the user may get used to the image, and the estimation accuracy of the facial expression and the psychological state may be lowered. Therefore, the state estimation device 1 may perform control to display different facial expression images at predetermined times. For example, the state estimation device 1 may perform control to display a positive image different from the positive image displayed last time. Note that the facial expression image may be a still image or a moving image.

In step S42, the state estimation device 1 images the face of the user viewing the facial expression image displayed in step S41, and acquires the face image.

In step S43, the state estimation device 1 estimates the facial expression of the user from the face image acquired in step S42. For example, the state estimation device 1 estimates scores (proportions) of “neutral, joy, surprise, sadness, and anger” indicated in the face image.

In step S44, the state estimation device 1 calculates the facial expression scores from the scores of the positive facial expressions (joy and surprise) and the scores of the negative facial expressions (anger and sadness) estimated in step S43.

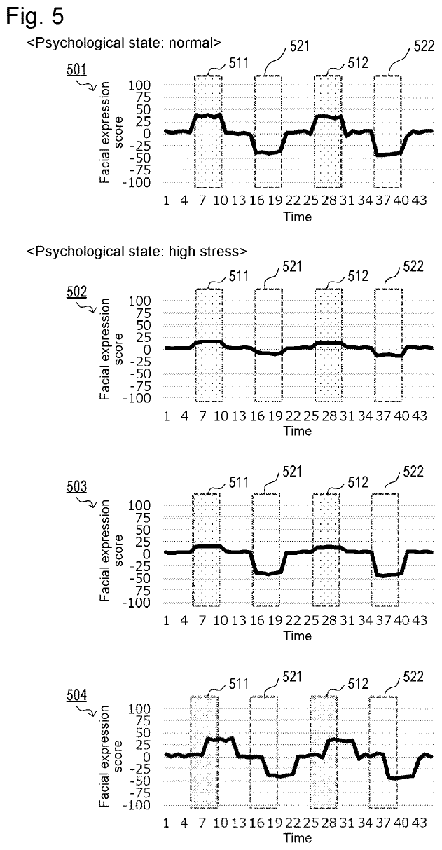

In step S45, the state estimation device 1 calculates a score feature that is a feature of a change in the facial expression score. Here, the change in the facial expression score will be described with reference to FIG. 5.

FIG. 5 is a diagram illustrating an example of the change in the facial expression score. Graphs 501 to 504 are graphs in which a horizontal axis is time and a vertical axis is a facial expression score. The graph 501 represents an example in a case where the psychological state of the user is a normal state. The graphs 502 to 504 represent examples of a case where the psychological state of the user is a high stress state. Periods 511 and 512 are periods corresponding to display periods in which the positive image is displayed in the facial expression image. Periods 521 and 522 are periods corresponding to display periods in which the negative image is displayed. Period other than the periods 511, 512, 521, and 522 are periods corresponding to non-display periods in which the facial expression image is not displayed.

For example, at the time of high stress, it is assumed that the activity of facial expression muscles is suppressed and the facial expression change becomes poor as compared with normal times. The graph 502 represents an example in which the change in the facial expression score is less than in the graph 501. In addition, at the time of high stress, it is assumed that a specific facial expression is amplified or suppressed as compared with normal times. The graph 503 represents an example in which the positive facial expression is suppressed (the facial expression scores in the periods 521 and 522 are small) as compared with the graph 501. In addition, at the time of high stress, it is assumed that the activity of the facial expression muscles is delayed as compared with normal times. The graph 504 represents an example in which the facial expression score changes later than the graph 501. It is assumed that the facial expression score changes in accordance with the psychological state as described above.

The state estimation device 1 calculates the score feature in order to evaluate such a change in the facial expression score. The score feature is, for example, a waveform pattern indicating a temporal change in the facial expression score in a predetermined period. For example, a shape of a waveform itself may be grasped as the score feature by using a model that handles time-series data such as GBDT. The predetermined period may be, for example, a waveform pattern of all the periods in which the face image is acquired. In addition, for example, the predetermined period may be a specific period such as one minute (for example, from 1 minute before the period 511 to the first 1 minute of the period 511) before and after the facial expression image is displayed, a display period and a non-display period (for example, after the period 511, a period including the period 521) of the display image, and the like.

In addition, for example, the score feature may be an average value of the facial expression scores at the time of facial expression imitation (a period corresponding to the display period of the facial expression image). The average value may be, for example, an average value in a period in which the positive image and the negative image are displayed, or an average value in one of the periods. In addition, in a case where there is a plurality of periods in which the positive image is displayed, an average value of the combined periods (for example, the period 511 and the period 512) may be used as the score feature.

In addition, for example, the score feature may be the amount of change in the facial expression score from the time of non-facial-expression imitation (period corresponding to the non-display period of the facial expression image) to the time of facial expression imitation. For example, in the example of the graph 501, the score feature may be a difference between an average value of facial expression scores in periods other than the periods 511, 512, 521, and 522 (at the time of facial expression imitation) and an average value of non-facial-expression scores in the periods 511 and 512 (at the time of facial expression imitation).

In addition, for example, the score feature may be a variance value of the facial expression scores at the time of non-facial-expression imitation and the facial expression scores at the time of facial expression imitation. For example, the variance value of the facial expression scores in the period before the positive image is displayed and the period (for example, the period 511) in which the positive image is displayed may be used. Note that the score feature is not limited thereto, and any feature that can evaluate the change in the facial expression score may be used.

The description returns to FIG. 4. In step S46, the state estimation device 1 estimates, by the state estimation unit 227, the psychological state of the user from the score feature calculated in step S45. For example, in a case where the change in the waveform calculated as the score feature is less than in normal times, the state estimation device 1 may estimate the high stress state. In addition, for example, the state estimation device 1 may estimate the high stress state in a case where the facial expression score at the time of facial expression imitation of the negative image calculated as the score feature is amplified as compared with normal times. Note that the state estimation device 1 may estimate whether the facial expression is “normal state” or “high stress state”, or may estimate “degree of stress of n %”. In a case where the facial expression score is estimated as “degree of stress of n %”, for example, a score feature at predefined “degree of stress of m %” may be compared with a current score feature, and the score feature may be calculated from a degree to which the change in the facial expression becomes poor or delayed.

For example, an example of processing in a case where the state estimation unit 227 estimates a psychological state of a user A by using the state estimation dictionary 228 will be described. Here, it is assumed that the state estimation dictionary 228 is a trained model in which the tendency of the user A is trained in advance by deep learning or the like. First, the state estimation unit 227 identifies (specifies) a user who estimates the psychological state. A method for identifying the user may be any method as long as the state estimation device 1 recognizes “who is the user”, and may be, for example, a method for performing individual identification from the face image, a method for allowing the user to input his or her ID, or the like. In the method for allowing the user to input his or her ID, the user may manually input the ID on a touch panel or the like, or may allow a reader to read an ID card (for example, an employee ID card). Subsequently, the state estimation unit 227 extracts a dictionary (state estimation dictionary 228 or trained model) of the user A. Subsequently, the state estimation unit 227 inputs data (for example, score feature) measured this time to the dictionary of the user A. Subsequently, the state estimation unit 227 acquires a psychological state that is an output of the dictionary of the user A.

In addition, for example, an example of processing in a case where the state estimation unit 227 estimates the psychological state of the user by a rule-based inference unit will be described. In this case, for example, the following plurality of dictionaries are prepared. A dictionary A is a dictionary that outputs “degree of stress (index indicating certainty of high stress)” from “average value of facial expression scores in display periods of positive image”. A dictionary B is a dictionary that outputs “degree of stress” from “display period of facial expression image and time lag of change in facial expression score”. A dictionary C is a dictionary that outputs “stress degree” from “difference between facial expression score in display period of positive image and facial expression score in display period of negative image”. The state estimation unit 227 may estimate the degree of stress (psychological state) by using any one of the dictionaries A to C. In addition, the state estimation unit 227 may integrate the degrees of stress calculated by the dictionaries A to C (for example, an average value, a maximum value, or the like) and output a final degree of stress.

In step S47, the state estimation device 1 outputs the psychological state. An output destination may be a device used by the user or a device (external server or the like) different from the device used by the user. In addition, the state estimation device 1 may output only the estimation result of the psychological state (for example, degree of stress of n %) or may output the facial expression estimation result (proportion of each expression, facial expression score, and the like).

Note that, in the example of FIG. 5, a case where the positive image and the negative image are alternately displayed has been described as an example, but a method for displaying each image is not limited thereto. For example, the period in which the positive image is displayed may be consecutive twice, or the period in which the positive image is displayed may be longer or shorter.

In addition, in the example in which the psychological state is estimated by using the trained model, it has been described that the input data is the score feature. Note that, depending on the design of the trained model, the time series data (waveform) of the facial expression score may be input to the trained model as the input data, and the psychological state may be obtained as the output.

The state estimation software described above is used, and thus, the user can easily and accurately estimate the psychological state.

### <Others>

The above embodiment merely describes, as examples, the configuration example of the present invention. The present invention is not limited to the specific forms described above, and various modifications can be made within the scope of the technical idea. For example, in the above embodiment, the example of estimating the psychological state of the user by the state estimation software has been described, but the application example of the present invention is not limited thereto.

### APPENDIX

A psychological state estimation device (1) includes a display section (20) configured to display a predetermined image (13), an imaging section (21) configured to image a face of an observer (11) viewing the predetermined image (13) displayed by the display section, a facial expression estimation section (222) configured to estimate a facial expression of the observer (11) from an image (12) of the face imaged by the imaging section (21), and a state estimation section (227) configured to estimate a psychological state of the observer (11) based on a change in the facial expression of the observer (11) viewing the predetermined image (13), the facial expression being estimated by the facial expression estimation section (222).

### DESCRIPTION OF SYMBOLS

1 state estimation device
11 user
12 face image
13 facial expression image

1. A psychological state estimation device comprising:
a display section configured to display a predetermined image;
an imaging section configured to image a face of an observer viewing the predetermined image displayed by the display section;
a facial expression estimation section configured to estimate a facial expression of the observer from an image of the face imaged by the imaging section; and
a state estimation section configured to estimate a psychological state of the observer based on a change in the facial expression of the observer viewing the predetermined image, the facial expression being estimated by the facial expression estimation section.

2. The psychological state estimation device according to claim 1, wherein the state estimation section is configured to analyze a correlation between the change in the facial expression and the psychological state, the state estimation section being configured to estimate the psychological state of the observer.

3. The psychological state estimation device according to claim 2, wherein the state estimation section is configured to analyze the correlation for each observer.

4. The psychological state estimation device according to claim 1, wherein the facial expression estimation section is configured to calculate a facial expression score obtained by quantifying the facial expression.

5. The psychological state estimation device according to claim 4, wherein the state estimation section is configured to estimate the psychological state based on a temporal change of the facial expression score in a predetermined period.

6. The psychological state estimation device according to claim 4, wherein the state estimation section is configured to estimate the psychological state based on an average value of the facial expression scores in periods corresponding to display periods in which the predetermined image is displayed.

7. The psychological state estimation device according to claim 4,
wherein the imaging section is configured to image the face in periods corresponding to display periods in which the predetermined image is displayed and the face in periods corresponding to non-display periods in which the predetermined image is not displayed, and
the state estimation section is configured to estimate the psychological state based on a change between facial expression scores in the periods corresponding to the display periods and facial expression scores in the periods corresponding to the non-display periods.

8. The psychological state estimation device according to claim 1, wherein the display section is configured to display different images at predetermined times.

9. The psychological state estimation device according to claim 1, wherein the predetermined image includes a positive image for inducing a positive emotion to the observer and a negative image for inducing a negative emotion to the observer.

10. The psychological state estimation device according to claim 1, further comprising:
an output section configured to output information indicating one or more emotions based on the psychological state estimated by the state estimation section.

11. A psychological state estimation method comprising:
a display step of displaying a predetermined image;
an imaging step of imaging a face of an observer viewing the predetermined image displayed in the display step;
a facial expression estimation step of estimating a facial expression of the observer from an image of the face imaged in the imaging step; and
a state estimation step of estimating a psychological state of the observer based on a change in the facial expression estimated in the facial expression estimation step.

12. A non-transitory computer readable medium storing a program for causing a computer to execute steps of a psychological state estimation method comprising:
a display step of displaying a predetermined image;
an imaging step of imaging a face of an observer viewing the predetermined image displayed in the display step;
a facial expression estimation step of estimating a facial expression of the observer from an image of the face imaged in the imaging step; and
a state estimation step of estimating a psychological state of the observer based on a change in the facial expression estimated in the facial expression estimation step.
